(19) United States
(12) Patent Application Publication
(10) Pub. No.: US 2008/0260806 A1
(43) Pub. Date: Oct. 23, 2008

(54) Personal hygiene compositions and methods

(76) Inventor: Risa Miller, Royal Oak, MI (US)

Correspondence Address: HARNESS, DICKEY & PIERCE, P.L.C., P.O. BOX 828, BLOOMFIELD HILLS, MI, 48303, US

(21) Appl. No.: 11/788,982

(22) Filed: Apr. 23, 2007

(51) Int. Cl. A61K 9/70; A61K 36/14; A61K 36/185; A61K 36/48; A61P 31/00; A61P 15/12; A61K 36/71; A61K 36/886

(52) U.S. Cl. 424/443; 424/726; 424/744; 424/757; 424/770; 424/773; 424/774; 424/775

(57) ABSTRACT

A personal hygiene composition, which may be coated on a towelette, comprises an astringent, a humectant, and a botanical extract. In some embodiments, the botanical extract can be selected from the group consisting of Hydrastis Canadensis, Tabebuia Impetiginosa, Hymenaea Courbaril, Ptychopetalum Olacoides, Juniperus Brasiliensis, Hamamelis Virginiana, and mixtures thereof. The composition may be used for personal hygiene, including for cleansing the skin of a human or animal. For example, the personal hygiene towelette may be used as a male hygiene product for cleansing of the penis and genital area.

### INTRODUCTION

The present technology generally relates to liquid personal cleansing compositions which, in some embodiments, are coated on a disposable towelette or wipe.

A wide variety of compositions and materials are known in the cosmetic and pharmaceutical arts for application to the skin. These compositions may be used, for example, for removing sebum, bacteria, dirt and other materials from the skin, or for depositing moisturizing, cosmetic, nutritional or pharmaceutical actives. Depending on the particular use, they may contain a wide variety of materials. Moreover, such compositions may be applied in a variety of ways, including by direct application as a liquid, by spray, or by using a towelette or similar substrate.

Formulating skin care products requires, however, balancing delivery of the desired cosmetic or therapeutic benefit, with possible undesirable effects. For example, many formulations are associated with skin irritation or other side effects. Others have poor aesthetic qualities or undesirable application characteristics.

Many skin care products are adapted for use in cleaning the genital area, in both men and women. Such products may be for cleansing, or may offer cosmetic or therapeutic benefits. However, because the skin in the genital area may be sensitive, many such compositions exhibit undesirable features.

For example, many products that contain antimicrobial agents are harsh or irritating to the skin due to the nature of the chemicals utilized to provide the antimicrobial effect. Some cleansers and surgical disinfectants utilize high levels of alcohol and/or surfactants which have been repeatedly shown to dry out and irritate skin tissues. Other products utilize harsh cationic surfactants. While such surfactants may be capable of penetrating and killing many types of bacteria, they may also be very irritating and harsh to the skin. Yet other antimicrobial-containing products utilize an organic acid in combination with an anionic or cationic surfactant as an antimicrobial agent. Although some organic acids can safely be used in products to control microorganisms without the presence of surfactants, most products incorporating only an organic acid have a low efficacy against bacteria and fungi unless used at very high concentrations. At very high concentrations, these acids can make the ultimate product uneconomical and can raise skin irritation concerns.

As such, a need continues to exist for personal care formulations, particularly for use in the genital area, that have acceptable characteristics. Ideally, such products would gently clean and deodorize the skin while significantly reducing the amount of active microbial agents, while at the same time not drying out or irritating the skin.

### SUMMARY

The present technology provides personal hygiene compositions, particularly for use in providing cleansing and other benefits to skin in the genital area. Topical compositions include those comprising an astringent, a humectant, and a botanical extract. The extract preferably comprises a mixture of at least three of Hydrastis Canadensis, Tabebuia Impetiginosa, Ptychopetalum Olacoides, Juniperus Brasiliensis, and Hamamelis Virginiana. The composition preferably has a pH of from about 4.0 to about 6.0. In various embodiments, such compositions further comprise at least one of an extract of Hymenaea Courbari, tocopherol, an extract of Aloe Barbadensis, and an essential oil. Such compositions may provide antimicrobial, antifungal, anti-inflammatory, or sexual stimulant benefits.

The present technology also provides personal hygiene towelettes comprising:

(a) a towelette substrate; and

(b) a composition coating a surface of said substrate, said composition comprising an astringent, a humectant, and a botanical extract selected from the group consisting of Hydrastis Canadensis, Tabebuia Impetiginosa, Hymenaea Courbaril, Ptychopetalum Olacoides, Juniperus Brasiliensis, Hamamelis Virginiana, and mixtures thereof,

wherein said composition has a pH of from about 4.0 to about 6.0. Preferably the botanical extract botanical extract comprises a mixture of at least three of Hydrastis Canadensis, Tabebuia Impetiginosa, Ptychopetalum Olacoides, Juniperus Brasiliensis, and Hamamelis Virginiana, and may further comprise an extract of Hymenaea Courbaril.

The present technology also provides methods of relieving irritation of a foreskin area of a penis. Methods also include those for treating a foreskin area of a penis with an antimicrobial, antifungal, and/or anti-bacteria component on a towelette.

### DESCRIPTION

The following description of the present technology is merely exemplary in nature of the subject matter, manufacture and use of one or more inventions, and is not intended to limit the scope, application, or uses of any specific inventions claimed in this application or in such other applications as may be filed claiming priority to this application, or patents issuing therefrom. The following definitions and non-limiting guidelines must be considered in reviewing the description of the technology set forth herein.

The headings (such as “Introduction” and “Summary”) and sub-headings used herein are intended only for general organization of topics within the present technology, and are not intended to limit the disclosure of the technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The citation of references herein or during prosecution of this application does not constitute an admission that those references are prior art or have any relevance to the patentability of the technology disclosed herein. All references cited in the “Description” section of this specification are hereby incorporated by reference in their entirety, for all purposes. In the event that one or more of the incorporated references, literature, and similar materials differs from or contradicts this application, including but not limited to defined terms, term usage, described techniques, or the like, this application controls.

The description and specific examples, while indicating some embodiments of the present technology, are intended for purposes of illustration only and are not intended to limit the scope of the technology disclosed herein. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, and methods of the present technology. Similarly, the terms “can” and “may” and their variants are intended to be non-limiting, such that recitation that an embodiment can or may comprise certain elements or features does not exclude other embodiments of the present technology that do not contain those elements or features.

The present technology provides compositions and methods for the application to skin in humans or other animals. Accordingly, specific compounds, materials and compositions to be used in this technology must, accordingly, be pharmaceutically or cosmetically acceptable. As used herein, such a “pharmaceutically acceptable” or “cosmetically acceptable” component is one that is suitable for use with humans and/or animals without undue adverse side effects (such as toxicity, irritation, and allergic response) commensurate with a reasonable benefit/risk ratio.

### Liquid Compositions

The present technology provides liquid compositions comprising an astringent, a humectant, and a botanical extract. In various embodiments, such compositions have antimicrobial efficacy. The compositions can be applied directly to the skin, or can be applied to the skin using a variety of substrates or carriers such as cellulosic webs, nonwoven structures, and textile-based materials. In one embodiment, a composition is coated on one or more surfaces of a wipe or towelette, such as is used in personal hygiene products among those known in the art. In various embodiments, the present technology provides antimicrobial formulations which are highly effective against a broad range of microorganisms such as bacteria, fungi, yeasts, molds, protozoan, and viruses.

The compositions of the present technology comprise at least one botanical extract. Preferably, the composition comprises a botanical extract selected from the group consisting of Hydrastis Canadensis, Tabebuia Impetiginosa, Hymenaea Courbaril, Ptychopetalum Olacoides, Juniperus Brasiliensis, Hamamelis Virginiana, and mixtures thereof. Preferably the botanical extract comprises at least three of Hydrastis Canadensis, Tabebuia Impetiginosa, Hymenaea Courbaril, Ptychopetalum Olacoides, Juniperus Brasiliensis, Hamamelis Virginiana. In some embodiments, the extract has antimicrobial efficacy, and is selected from the group consisting of Hydrastis Canadensis, Tabebuia Impetiginosa, Hymenaea Courbaril, Aloe Barbadensis, and mixtures thereof. In some embodiments, the compositions provide a sexual stimulant benefit, such as treating erectile dysfunction in a human male, and comprise a botanical extract selected from the group consisting of Juniperus Brasiliensis, Ptycohopetalum Olacoides, and mixtures thereof. The botanical extract can be present in an amount from about 1% to about 50% and, preferably from about 10% to about 40%. In one embodiment the extract is present at a level of about 20%.

The extracts of the present technology comprise any material derived from the plant materials discussed herein, including naturally-derived and synthetic compounds and mixtures of compounds. In some embodiments, the extracts are naturally derived using extraction techniques among those known in the art. For example, an extract may be made by heating (e.g., boiling) plant material in water followed by filtering. In some embodiments, the plant material is boiled in water, allowed to steep for a period of time (e.g., 2, 4, 6, 8 or more hours), heated again, then filtered to remove some or all solids. The final extract may comprise from about 20% to 50% of plant derived compounds in a water solvent.

The compositions also comprise an astringent. The astringent may be a botanical extract, such as Hamamelis Virginiana or Citrus Limon. In one embodiment, the astringent is a witch hazel extract of Hamamelis Virginiana in a solution comprising about 14% alcohol. The astringent may be present at a level of from about 10% to about 80%, from about 20% to about 60%, or from about 30% to about 50%. In one embodiment, the astringent is present at a level of about 48%.

The compositions of the present technology may comprise a humectant. Humectants among those useful herein are selected from the group consisting of glycerol, propylene glycol, glycerin, sorbitol, urea, amino acids, polyol, aloe, tocopherol, other compounds with hygroscopic properties, and mixtures thereof. A preferred humectant is selected from the group consisting of glycerin, aloe, tocopherol, and mixtures thereof. The humectant may be present in an amount of from about 0.1% to about 25%, or from about 1% to about 10%. In one embodiment, the humectant is present at a level of about 5%.

The formulation may optionally comprise a preservative. Examples of preservatives useful herein include sodium hydroxymethylglycinate, polyaminopropyl biguanide, quaternary ammonium compound, EDTA salt, EDTA fatty acid conjugate, alkanol especially ethanol, isopropyl alcohol, benzyl alcohol, paraben, sorbate, diazolidinyl urea, iodopropynyl butylcarbamate, urea derivative, and isothiazolinone, and mixtures thereof. A preferred preservative can be sorbate. The preservative can be present in an amount from about 0.2 wt % to about 2 wt %, and more preferably about 0.5 wt %, of the total weight of the formulation.

The compositions of the present technology can optionally comprise an essential oil. Examples of essential oils useful herein include Anise Oil, Balm Mint Oil, Basil Oil, Bee Balm Oil, Bergamot Oil, Birch Oil, Bitter Almond Oil, Bitter Orange Oil, Calendula Oil, California Nutmeg Oil, Caraway Oil, Cardamom Oil, Chamomile Oil, Cinnamon Oil, Clary Oil, Cloveleaf Oil, Clove Oil, Coriander Oil, Cypress Oil, Eucalyptus Oil, Fennel Oil, Gardenia Oil, Geranium Oil, Ginger Oil, Grapefruit Oil, Hops Oil, Hyptis Oil, Indigo Bush Oil, Jasmine Oil, Juniper Oil, Kiwi Oil, Laurel Oil, Lavender Oil, Lemongrass Oil, Lemon Oil, Linden Oil, Lovage Oil, Mandarin Orange Oil, Matricaria Oil, Musk Rose Oil, Nutmeg Oil, Olibanum, Orange Flower Oil, Orange Oil, Patchouli Oil, Pennyroyal Oil, Peppermint Oil, Rose Hips Oil, Rosemary Oil, Rose Oil, Rue Oil, Sage Oil, Sambucus Oil, Sandalwood Oil, Sassafras Oil, Silver Fir Oil, Spearmint Oil, Sweet Marjoram Oil, Sweet Violet Oil, Tea Tree Oil, Thyme Oil, Wild Mint Oil, Yarrow Oil, Ylang Ylang Oil, and mixtures thereof. A preferred essential oil is Lemon Oil. The essential oil can be present in an amount from about 0.1% to about 5%. In one embodiment, the essential oil is present at a level of about 0.15%.

In some embodiments, the compositions can comprise a fragrance, which may be an essential oil as discussed above. Appropriate fragrances useful herein include those known in the art.

The compositions of the present technology can optionally comprise a surfactant. For example, suitable surfactants may include anionic surfactants, nonionic surfactants, cationic surfactants, amphoteric surfactants, and mixtures thereof. In embodiments where the composition is coated on a towelette or similar substrate, the nature and amount of surfactant should be selected so as to not cause excessive foaming of the composition. Suitable anionic surfactants include sodium laureth sulfate, sodium-lauryl methyl taurate, myristoyl sarcosinate, sodium dodecylbenzene sulfonate, and the like. Suitable nonionic surfactants include the polyoxyethylene ethers of the higher fatty alcohols and alkyl phenols; the polyethylene glycols of fatty acids; fatty alkylol amide condensation products; polymers of ethylene and propylene oxides; compounds formed by the addition of propylene oxide to ethylene diamide, followed by the addition of ethylene oxide; fatty acid ethylene oxide condensation products; ethoxylate carboxylic acid; ethoxylate glycerides; and glycol esters.

In some embodiments, the composition comprises a solvent, such as alcohol (e.g, ethanol and isopropanol), water, and mixtures thereof. Water may be present, for example, at a level of from about 5% to about 80%. The solvent may be a component in the botanical blend or in any of the other components used in the formulation.

In some embodiments of the present technology, the formulation comprises a buffer or pH adjuster, such as citric acid. In various embodiments, the formulation can have a pH of from about 2 to about 10, from about 3 to about 7, from about 4 to about 6.5, from 4 to about 6, from about 5.2 to about 5.9, from about 5.4 to about 5.9, and from about 5.4 to about 5.6.

### Towelettes

The compositions of the present technology can be incorporated into a towelette, such as are used for wet wipes, hand wipes, face wipes, cosmetic wipes, household wipes, and industrial wipes among those known in the art. Materials suitable for the substrate of towelettes include materials known in the art, and are typically made from a fibrous sheet material which may be either woven or nonwoven. For example, nonwoven fibrous sheet materials include meltblown, coform, air-laid, bonded-carded web materials, hydroentangled materials, and combinations thereof. Such materials can be comprised of synthetic or natural fibers, or combinations thereof. Typically, towelettes have a basis weight of from about 25 to about 120 grams per square meter, or from about 40 to about 90 grams per square meter.

In some embodiments, the towelettes comprise a coform basesheet of polymeric microfibers and cellulosic fibers having a basis weight of from about 60 to about 80 grams per square meter and desirably about 75 grams per square meter. Typically, such coform base sheets comprise a gas-formed matrix of thermoplastic polymeric meltblown microfibers, such as, for example, polypropylene microfibers, and cellulosic fibers, such as, for example, wood pulp fibers.

The relative percentages of the polymeric microfibers and cellulosic fibers in the coform basesheet can vary over a wide range depending upon the desired characteristics of the towelettes. For example, the coform basesheet may comprise from about 20 to about 100 weight percent, desirably from about 20 to about 60 weight percent, and more desirably from about 30 to about 40 weight percent of the polymeric microfibers based on the dry weight of the coform basesheet being used to provide the towelettes.

The towelette of the present technology can comprise a composite which includes multiple layers of materials. For example, the towelettes may include a three layer composite which includes an elastomeric film or meltblown layer between two coform layers as described above. In some embodiments, the coform layers may define a basis weight of from about 15 to about 30 grams per square meter and the elastomeric layer may include a film material such as a polyethylene metallocene film.

The towelettes of the present technology comprise a composition of the present technology, as discussed above, coated on a surface of the towelette substrate. Such “coating on” includes coating on, or into, a portion of one surface, on an entire surface, or both planar surfaces of the substrate of the towelette. The amount of composition coated may vary depending upon the chemical composition and construction of the substrate, the materials comprising the composition, the type of container being used to store the towelettes, and the desired end use of the towelettes. The specific amount of composition coated is selected, for example, so as that the towelette is not too dry and may not adequately perform. On the other hand, if the amount of composition coated is too high, the towelette may be oversaturated and soggy so as to lose physical integrity during use, and the composition may pool in the bottom of the container holding the towelettes. In various embodiments, a towelette can contain from about 150 to about 600 weight percent and desirably from about 250 to about 450 weight percent solution based on the dry weight of the wipe for improved wiping. In some embodiments, wherein the towelettes are made from a coform material comprising from about 30 to about 40 weight percent polymeric microfibers based on the dry weight of the wipe, the amount of composition coated on the towelette is from about 300 to about 400 weight percent and desirably about 330 weight percent based on the dry weight of the towelette.

The coated towelettes may be packaged in a sealed foil packet or other package suitable for retaining moisture in the coated towelettes. The sealed package may contain one towelette, for individual single use, or multiple towelettes. Suitable packaging materials include those known in the art.

### Methods

The compositions and personal hygiene towelettes of the present technology may be used for personal hygiene, including for cleansing the skin of a human or animal or other cosmetic purposes. In some embodiments, the compositions and the personal hygiene towelettes may be used to gently clean and deodorize the skin while significantly reducing the amount of active microbial agents, while at the same time not drying out or irritating the skin.

For example, a personal hygiene towelette may be used as a male hygiene product for cleansing of the penis and genital area. This may be particularly useful for uncircumcised men since its use may combat microbial infections underneath the foreskin. In some embodiments, the present technology provides methods for treating or preventing disorders associated with the male genitals, particularly those having a microbial etiology, including phimosis and balanoposthitis. Methods can include those for relieving irritation in the foreskin area of a penis.

In some embodiments, the present technology also provides methods for sexual stimulation comprising applying a composition of the present technology, optionally delivered as a towelette, to the penis. Methods can include those for enhancing arousal, enhancing an erection, and treating or preventing erectile dysfunction.

Some embodiments and the examples described herein are exemplary and not intended to be limiting in describing the full scope of compositions and methods of these technology. Equivalent changes, modifications and variations of some embodiments, materials, compositions and methods can be made within the scope of the present technology, with substantially similar results.

1. A personal hygiene towelette comprising:
a towelette substrate; and
a composition coating a surface of said substrate, said composition comprising an astringent, a humectant, and a botanical extract selected from the group consisting of Hydrastis Canadensis, Tabebuia Impetiginosa, Hymenaea Courbaril, Ptychopetalum Olacoides, Juniperus Brasiliensis, Hamamelis Virginiana, and mixtures thereof,
wherein said composition has a pH of from about 4 to about 6.5.

2. A towelette according to claim 1, wherein said botanical extract comprises a mixture of at least three of Hydrastis Canadensis, Tabebuia Impetiginosa, Ptychopetalum Olacoides, Juniperus Brasiliensis, and Hamamelis Virginiana.

3. A towelette according to claim 1, wherein said botanical extract comprises Hydrastis Canadensis, Tabebuia Impetiginosa, Ptychopetalum Olacoides, Juniperus Brasiliensis, and Hamamelis Virginiana.

4. A towelette according to claim 2, wherein said botanical extract further comprises an extract of Hymenaea Courbaril.

5. A towelette according to claim 1 further comprising at least one of tocopherol, an extract of Aloe Barbadensis, and an essential oil.

6. A towelette according to claim 1, wherein said astringent is an extract of Hydrastis Canadensis.

7. A towelette according to claim 1 having one or more properties of antimicrobial, antifungal, anti-inflammatory, and sexual stimulant.

8. A towelette according to claim 1, wherein said pH is from about 5.4 to about 6.

9. A topical composition comprising:
an astringent,
a humectant, and
a botanical extract comprising a mixture of at least three of Hydrastis Canadensis, Tabebuia Impetiginosa, Ptychopetalum Olacoides, Juniperus Brasiliensis, and Hamamelis Virginiana,
wherein said composition has a pH of from about 4 to about 6.5.

10. A composition according to claim 9, wherein said botanical extract comprises Hydrastis Canadensis, Tabebuia Impetiginosa, Ptychopetalum Olacoides, Juniperus Brasiliensis, and Hamamelis Virginiana.

11. A composition according to claim 10, wherein said botanical extract further comprises an extract of Hymenaea Courbaril.

12. A composition according to claim 9 further comprising at least one of tocopherol, an extract of Aloe Barbadensis, and an essential oil.

13. A composition according to claim 9 having one or more properties of antimicrobial, antifungal, anti-inflammatory, and sexual stimulant.

14. A composition according to claim 9, wherein said pH is from about 5.4 to about 6.

15. A method of relieving irritation in a foreskin area of a penis, the method comprising cleansing said area with a composition comprising an astringent, and a botanical extract selected from the group consisting of Hydrastis Canadensis, Tabebuia Impetiginosa, Hymenaea Courbaril, Ptychopetalum Olacoides, Juniperus Brasiliensis, Hamamelis Virginiana, and mixtures thereof
wherein said composition has a pH of from about 4 to about 6.5.

16. A method according to claim 15, wherein said botanical extract comprises Hydrastis Canadensis, Tabebuia Impetiginosa, Ptychopetalum Olacoides, Juniperus Brasiliensis, and Hamamelis Virginiana.

17. A method according to claim 15, wherein said botanical extract further comprises an extract of Hymenaea Courbaril.

18. A method according to claim 15 further comprising at least one of tocopherol, an extract of Aloe Barbadensis, and an essential oil.

19. A method according to claim 15 having one or more properties of antimicrobial, antifungal, anti-inflammatory, and sexual stimulant.

20. A method according to claim 15, wherein said pH is from about 5.4 to about 6.

21. The method according to claim 15 wherein said composition is effective as an anti-infective.

22. A method according to claim 15, wherein said composition is effective as a sexual stimulant.

23. A method of treating erectile dysfunction in a human male comprising contacting the penis of said male with a towelette comprising a fabric substrate and a composition comprising an extract from at least one of Juniperus Brasiliensis and Ptychopetalum Olacoides.

24. A method according to claim 23, wherein said composition comprises a mixture of said extracts.

25. A method according to claim 23, wherein said composition further comprises an extract selected from the group consisting of Hydrastis Canadensis, Tabebuia Impetiginosa, Hymenaea Courbaril, Hamamelis Virginiana, and mixtures thereof.
